(19) United States
(12) Patent Application Publication
(10) Pub. No.: US 2016/0152679 A1
(43) Pub. Date: Jun. 2, 2016

(54) RELAXIN-LIKE COMPOUNDS AND USES THEREOF

(71) Applicant: ANGION BIOMEDICA CORP., Uniondale, NY (US)

(72) Inventors: Prakash Narayan, East Northport, NY (US); Rama K. Mishra, Chicago, IL (US); E. Siobhan McCormack, Glen Head, NY (US); Victor M. Garsky, Blue Bell, PA (US); Itzhak D. Goldberg, Englewood, NY (US)

(73) Assignee: ANGION BIOMEDICA CORP., UNIONDALE, NY (US)

(21) Appl. No.: 14/403,185

(22) PCT Filed: May 24, 2013
(86) PCT No.: PCT/US2013/042681

§ 371 Date: Nov. 23, 2014

Related U.S. Application Data
(60) Provisional application No. 61/651,517, filed on May 24, 2012.

(51) Int. Cl. C07K 14/64

(52) U.S. Cl. CPC C07K 14/64; A61K 38/00

(57) ABSTRACT

The present invention is directed to novel relaxin-like compounds having at least one intramolecular or intramolecular bridge that is a bioisosteric substitution of a cystine.

### INTRODUCTION

The present invention is directed to novel relaxin (H2R)-like peptides. The relaxin-like peptides may bind to the relaxin receptor RXFP1. In particular, the present invention is drawn to relaxin-like peptides that are acyclic or cyclic, optionally have one of more truncations of the A or B chains, and have at least one cystine replaced with a bioisosteric substitution.

The invention also encompasses methods for treating, preventing or ameliorating a disease or disorder and or treating, restoring or ameliorating a tissue injury using relaxin-like peptides of the current invention. The invention also encompasses methods for treatment of heart failure and liver, lung and kidney fibrosis, among other injuries and diseases.

### BACKGROUND OF THE INVENTION

Relaxin (H2R) is an ˜6 kd peptide hormone member of the insulin superfamily comprising a 24 amino acid A chain (also called A peptide) and a 29 amino acid B chain (also called B peptide) linked by disulfide bridges. The amino acid sequence of the H2R A peptide is ZLYSALANKCCHVGCTKRSLARFC (SEQ ID NO: 1). The amino acid sequence of the H2R B peptide is DSWMEEVIKLCGRELVRAQIAICGMSTWS (SEQ ID NO:2). In the native H2R molecule, an intramolecular cystine bridges the cysteines at positions 10 and 15 of the H2R A peptide, an intermolecular cystine bridges cysteine 11 of the H2R A peptide to cysteine 11 of the H2R B peptide, and a second intermolecular cystine bridges cysteine 24 of the H2R A peptide to cysteine 23 of the H2R B peptide.

Despite their structural similarity, H2R and insulin bind to distinct and unrelated receptors and, hence, have no common cellular effects. H2R couples with its receptor, RXFP1, a G-protein coupled receptor (GPCR), stimulating cellular production of cAMP and NO. Typically found in adult systemic circulation at miniscule concentrations, systemic H2R levels increase dramatically with pregnancy. Historically, this peptide been associated with ripening of the cervix for parturition, and softening of the birth canal which requires breakdown of profibrotic collagens. In fact, one of the most consistent biological effects of H2R is its ability to stimulate breakdown of profibrotic collagen not only in the birth canal but in other adult tissue affected by fibrosis. Supporting this notion, H2R-null mice exhibit progeria and advanced fibrosis of the heart, kidney and lung in addition to the reproductive tract. In most tissues, fibrosis was more pronounced in H2R-null male mice, which indicates that this peptide is relevant in nonreproductive tissues in males as well. Importantly, excess collagen accumulation was reversed by supplementing H2R in these animals. Together, these data suggest that H2R might be used therapeutically to reduce scarring caused by the accumulation of collagen in fibrotic diseases. Moreover, exogenous administration of H2R is therapeutic in preclinical models of systemic sclerosis (SSc), and in hepatic, renal pulmonary and cardiac fibrosis.

It is towards the identification of relaxin-like (H2R-like) peptides useful therapeutically for the aforementioned and other purposes that the present invention is directed.

### SUMMARY OF THE INVENTION

The present invention is directed to acyclic and cyclic compounds that comprise a modified H2R A peptide linked to a modified H2R B peptide. In one embodiment, the compound comprises an optionally truncated H2R A peptide chain and an optionally truncated H2R B peptide chain, and wherein one or more intramolecular or intramolecular cystines is replaced with a bioisosteric substitution. A bioisosteric substitution replaces a cystine bridge with a covalent, non-labile bridge and maintains at least one biological activity of the compound. In other embodiments, cysteine residues in the peptide that are not replaced with a bioisostere are replaced with another amino acid, such as but not limited to alanine. The compounds of the invention comprise at least a portion if not the entire sequence of the H2R A peptide, at least a portion if not the entire sequence of the H2R B peptide, and at least one intermolecular bridge there between, said intermolecular bridge being a bioisosteric substitution of the cysteine bridge, such as but not limited to 1) a diaminopropane molecule bound via amide bonds to the C-terminal carboxylic acids of the H2R A and B peptides, 2) an aspartic acid replacing one cysteine residue of a cystine bridge, and a L-2,3-diaminopropionic acid replacing the other cysteine residue of the cysteine bridge, the pendant amino group of the L-2,3-diaminopropionic acid and the carboxylic acid of the aspartic acid linked by an amide bond; 3) a propargylglycine replacing one cysteine residue of a cysteine bridge and a 2-amino-4-azidobutyric acid replacing the other cysteine residue of the cysteine bridge, the pendant groups of the two molecules forming a triazine ring; 4) the cystine bridge is replaced by cystathionine; or any combination of any of the foregoing. In other embodiments, one or more cysteine residues of either or both the native or truncated H2R A peptide and/or H2R B peptide are replaced with a different amino acid such as but not limited to alanine. In other embodiments, the N-terminal glutamine or glutamic acid is a pyroglutamate residue. In other embodiments, the compound of the invention may also have an intramolecular cysteine bridge replaced with a bioisosteric substitution, such as but not limited to those mentioned above, and in another embodiment, the bioisosteric substitution is cystathionine. In other embodiments, a diaminopropane, propargylglycine or 2-amino-4-azidobutyric acid can be inserted into the amino acid sequence to provide one member of an intramolecular or intermolecular bridge.

Non-limiting examples of such compounds include compounds 1-10 below:

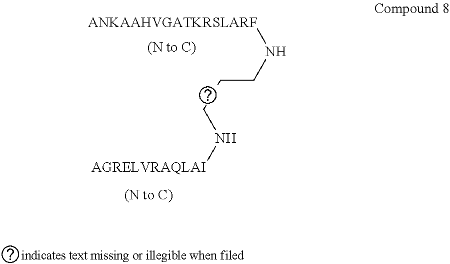

wherein the C-terminal carboxylic acids of the Phe of the H2R A peptide and the C-terminal lie of the H2R B peptide are linked through amide bonds with 1,3-diaminopropane;

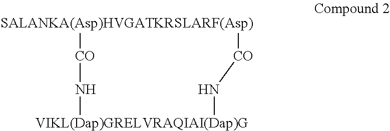

wherein Dap represents L-2,3-diaminopropionic acid;

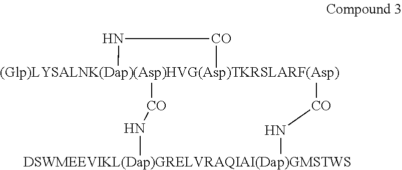

wherein Dap represents L-2,3-diaminopropionic acid and Glp represents pyroglutamate;

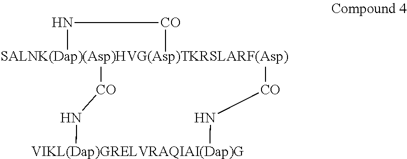

wherein Dap represents L-2,3-diaminopropionic acid;

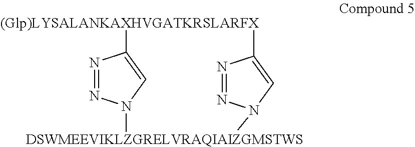

wherein Glp is pyroglutamate, X is propargylglycine and Z is 2-amino-4-azidobutyric acid, or Z is propargylglycine and X is 2-amino-4-azidobutyric acid;

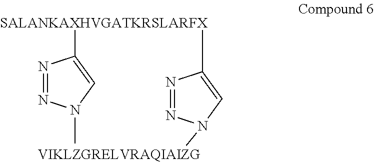

wherein X is propargylglycine and Z is 2-amino-4-azidobutyric acid, or Z is propargylglycine and X is 2-amino-4-azidobutyric acid;

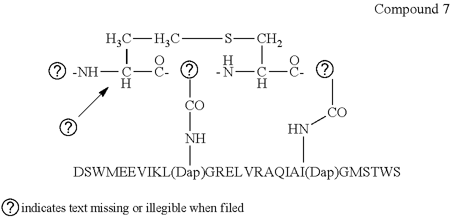

wherein Z is Glu or Gln;

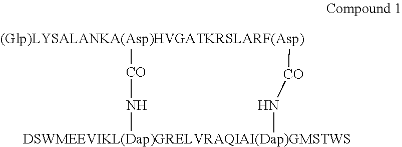

wherein Dap represents L-2,3-diaminopropionic acid;

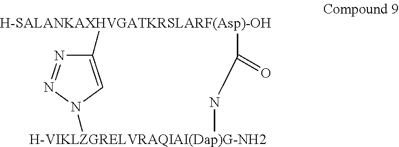

wherein Dap represents L-2,3-diaminopropionic acid, X is propargylglycine and Z is 2-amino-4-azidobutyric acid; and

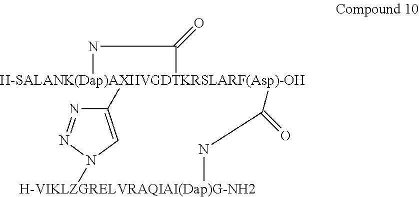

wherein Dap represents L-2,3-diaminopropionic acid, X is propargylglycine and Z is 2-amino-4-azidobutyric acid.

In certain embodiments, one or more amino acid in either the A or B peptide or both is replaced with a conservative or a non-conservative substitution.

In other embodiments, the H2R A or B peptide has less than 90%, less than 85%, less than 80%, less than 75%, less than 70%, less than 65%, less than 60%, less than 55%, less than 50%, less than 45%, less than 40%, less than 35%, less than 30%, or less than 20 percent sequence identity with any portion of the amino acid sequence of either the native H2R A peptide or the H2R B peptide of human relaxin, or of both peptides.

In further embodiments, compositions including pharmaceutical compositions of the aforementioned compounds are embraced herein. In other embodiments, the invention also encompasses methods for treating, preventing or ameliorating a disease or disorder or treating, restoring or ameliorating a tissue injury using relaxin-like peptides of the current invention. The invention also encompasses methods for treatment of heart failure and liver, lung and kidney fibrosis, among other injuries and diseases.

### TERMINOLOGY

As used herein, the terms “about” or “approximately” when used in conjunction with a number refer to any number within 1, 5, or 10% of the referenced number.

The term “administered in conjunction with” in the context of the methods of the invention means administering a compound prior to, at the same time as, and/or subsequent to the onset of a disease, disorder, or condition.

The term “amino acid” or any reference to a specific amino acid is meant to include naturally occurring proteogenic amino acids as well as non-naturally occurring amino acids such as amino acid analogs. Those skilled in the art would know that this definition includes, unless otherwise specifically noted, includes naturally occurring protogenic (L)-amino acids, their optical (D)-isomers, chemically modified amino acids, including amino acid analogs such as penicillamine (3-mercapto-D-valine), naturally occurring non-proteogenic amino acids such as norleucine and chemically synthesized proteins that have properties known in the art to be characteristic of an amino acid. As used herein, amino acids will be represented wither by their three letter acronym or one letter symbol as follows: alanine-Ala or A, arginine-Arg or R, asparagine=Asn or N, aspartic acid=Asp or D, cysteine=Cys or C, glutamic acid=Glu or E, glutamine=Gln or Q, glycine=Gly or G, histidine=His or H, isoleucine=Ile or I, leucine=Leu or L, lysine=Lys or K, methionine=Met or M, phenylalanine=Phe or F, proline=Pro or P, serine=Ser or S, threonine=Thr or T, tryptophan=Trp or W, tyrosine=Tyr or Y, and valine=Val or V. Glp refers to pyroglutamic acid. Z when located at the N-terminus of a peptide indicates it can be either Glu or Gln. Additionally, the term “amino acid equivalent” refers to compound that depart from the structure of the naturally occurring amino acids, but which have substantially the structure of an amino acid, such that they can be substituted within H2R A peptide or B peptide, which retains its biological activity despite the substitution. Thus, for example, amino acid equivalents can include amino acids having side chain modifications or substitutions, and also include related organic acids, amides or the like. The term “amino acid” is intended to include amino acid equivalents. The term “residues” refers both to amino acids and amino acid equivalents. Amino acids may also be classified into the following groups as is commonly known in the art: (1) hydrophobic amino acids: His, Trp, Tyr, Phe, Met, Leu, Ile, Val, Ala; (2) neutral hydrophilic amino acids: Cys, Ser, Thr; (3) polar amino acids: Ser, Thr, Asn, Gln; (4) acidic/negatively charged amino acids: Asp, Glu; (5) charged amino acids: Asp, Glu, Arg, Lys, His; (6) positively charged amino acids: Arg, Lys, His; and (7) basic amino acids: His, Lys, Arg.

An “isolated” or “purified” polypeptide is substantially free of cellular material or other contaminating proteins from the cell or tissue source from which the protein or polypeptide is derived, or substantially free of chemical precursors or other chemicals when chemically synthesized. The language “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptides having less than about 30%, 20%, 10%, or 5% (by dry weight) of heterologous protein (also referred to herein as a “contaminating protein”). When the polypeptide is recombinantly produced, it is also preferably substantially free of culture medium, i.e., culture medium represents less than about 20%, 10%, or 5% of the volume of the protein preparation. When the polypeptide is produced by chemical synthesis, it is preferably substantially free of chemical precursors or other chemicals, i.e., it is separated from chemical precursors or other chemicals which are involved in the synthesis of the protein. Accordingly such preparations of the polypeptide have less than about 30%, 20%, 10%, 5% (by dry weight) of chemical precursors or compounds other than the antibody of interest. In a preferred embodiment, polypeptides of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding a polypeptide of the invention is isolated or purified.

As used herein, the terms “peptide,” “polypeptide” and “protein” are used interchangeably and in their broadest sense to refer to constrained (that is, having some element of structure as, for example, the presence of amino acids which initiate a β turn or β pleated sheet, or for example, cyclized by the presence of disulfide bonded Cys residues) or unconstrained (e g, linear) amino acid sequences.

The term “preventing a disease, disorder, or condition” means delaying the onset, hindering the progress, hindering the appearance, protection against, inhibiting or eliminating the emergence, or reducing the incidence, of such disease, disorder, or condition. Use of the term “prevention” is not meant to imply that all patients in a patient population administered a preventative therapy will never develop the disease, disorder, or condition targeted for prevention, but rather that the patient population will exhibit a reduction in the incidence of the disease, disorder, or condition. For example, many flu vaccines are not 100% effective at preventing flu in those administered the vaccine. One skilled in the art can readily identify patients and situations for whom preventative therapy would be beneficial, such as, but not limited to, individuals about to engage in activities that may lead to trauma and injury (e.g., soldiers engaging in military operations, race car drivers, etc.), patients for whom surgery is planned, patients at risk for inherited diseases, disorders, or conditions, patients at risk for diseases, disorders, or conditions precipitated by environmental factors, or portions of the population at risk for particular diseases, disorders, or conditions such as the elderly, infants, or those with weakened immune systems, or those patients with genetic or other risk factors for a disease, disorder, or condition.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a non-primate (e.g., a monkey or a human), and more preferably a human.

To determine the percent identity of two amino acid sequences, the sequences are aligned for optimal comparison purposes. The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length. In an alternate embodiment, the sequences are of different length and, accordingly, the percent identity refers to a comparison of the shorter sequence to a portion of the longer sequence, wherein said portion is the same length as said shorter sequence.

### BRIEF DESCRIPTION OF THE FIGURES

FIG. 1 shows that a compound of the invention stimulates cAMP via RXFP1; and

FIG. 2 shows that a compound of the invention dose responsively stimulates cAMP production.

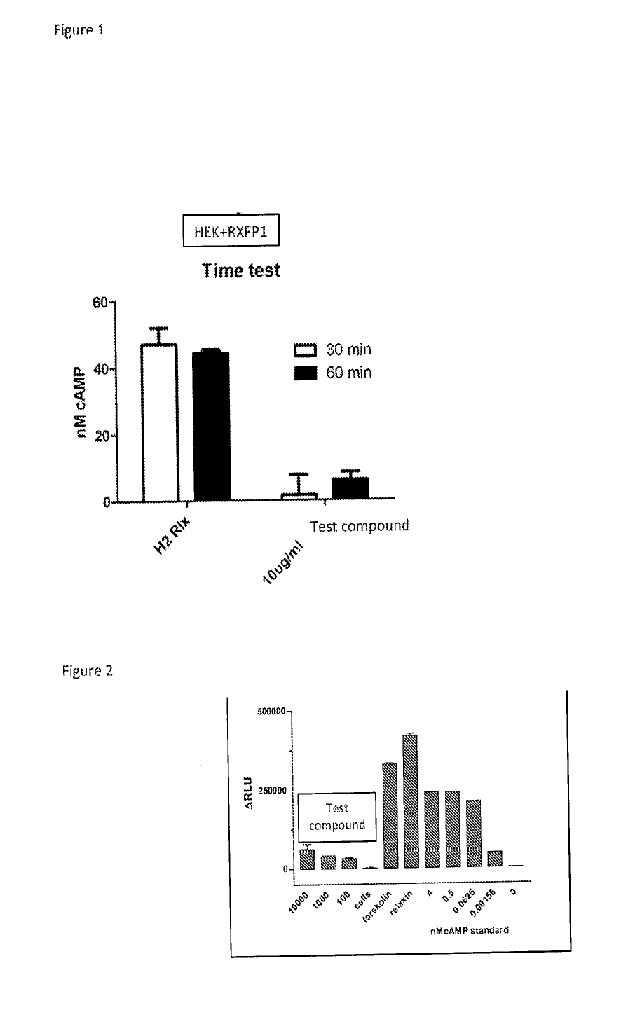

### DETAILED DESCRIPTION

The present invention is directed to acyclic and cyclic compounds that comprise a modified H2R peptide A linked to a modified H2R B peptide. In one embodiment, the compound comprises optionally truncated H2R peptide A and optionally truncated H2R B peptide chains, and wherein one or more intramolecular or intramolecular cystines is replaced with a bioisosteric substitution. In other embodiments, cysteine residues in the peptide that are not replaced with a bioisostere are replaced with another amino acid. As noted above, the amino acid sequence of the H2R A peptide is ZLYSALANKCCHVGCTKRSLARFC (SEQ ID NO: 1). The amino acid sequence of the H2R B peptide is DSWMEEVIKLCGRELVRAQIAICGMSTWS (SEQ ID NO:2). In the native H2R molecule, an intramolecular cystine bridges the cysteines at positions 10 and 15 of the H2R A peptide, and two intermolecular cystines bridge cysteine 11 of the H2R A peptide to cysteine 11 of the H2R B peptide, and bridge cysteine 24 of the H2R A peptide to cysteine 23 of the H2R B peptide. The compounds of the invention comprise at least a portion if not the entire sequence of the H2R A peptide, at least a portion if not the entire sequence of the H2R B peptide, and at least one intermolecular bridge there between, said intermolecular bridge being a bioisosteric substitution of the cysteine bridge, such as but not limited to 1) a diaminopropane molecule bound via amide bonds to the C-terminal carboxylic acids of the A and H2R B peptides, 2) an aspartic acid replacing one cysteine residue of a cystine bridge, and a L-2,3-diaminopropionic acid replacing the other cysteine residue of the cysteine bridge, the pendant amino group of the L-2,3-diaminopropionic acid and the carboxylic acid of the aspartic acid linked by an amide bond; 3) a propargylglycine replacing one cysteine residue of a cysteine bridge and a 2-amino-4-azidobutyric acid replacing the other cysteine residue of the cysteine bridge, the pendant groups of the two molecules forming a triazine ring; 4) the cystine bridge is replaced by cystathionine; or any combination of any of the foregoing. In other embodiments, one or more cysteine residues of either or both the native or truncated H2R A peptide and/or H2R B peptide are replaced with a different amino acid such as but not limited to alanine. In other embodiments, the N-terminal glutamine or glutamic acid is a pyroglutamate residue. In other embodiments, the compound of the invention may have an intramolecular cysteine bridge replaced with a bioisosteric substitution, such as but not limited to those mentioned above, and in another embodiment, the bioisosteric substitution is with cystathionine. In any of the foregoing, the amino acid residues contributing to the bioisosteric replacement bridge may be present in either orientation; i.e., where an Asp may replace a Cys in the A peptide and a Dap in the B peptide, in other embodiments, Dap replaces the Cys in the A peptide and Asp replaces Cys in the B peptide. In other embodiments, the A or B peptide or both may have a C-terminal amide, or the N-terminus may be acetylated.

In one embodiment, the compound has the structure of Compound 1 below:

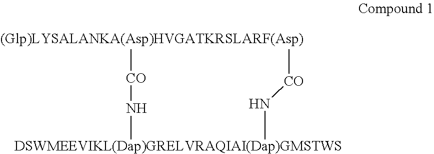

wherein Dap represents L-2,3-diaminopropionic acid. This compound comprises the H2R A peptide of H2R with an N-terminal Glp, Ala replacing Cys at positions 10 and 15, and Asp replacing Cys at positions 11 and 24. This compound also comprises H2R peptide B, the Cys at positions 11 and 23 are replaced with Dap, the 2-amino group and the carboxylic acid thereof peptide (amide) bound within the peptide chain. The pendant 3-amino groups of the Dap in the H2R B peptide and the carboxyl groups of the Asp in the H2R A peptide are bonded via amide bonds. In another embodiment, the orientation of one or both of the bioisosteric bridges are reversed; i.e., the Dap may be located in the H2R A peptide and the Asp in the H2R B peptide, for one or both bridges. In other embodiments, conservative or non-conservative amino acid substitutions of one or more residues in either the H2R A peptide, the H2R B peptide, or both, are provided.

In one embodiment, the compound has the structure of Compound 2 below:

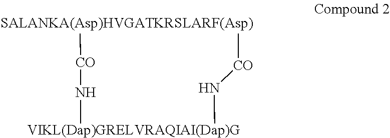

wherein Dap represents L-2,3-diaminopropionic acid. This compound comprises the A peptide of H2R with a deletion of the first three amino acids, Ala replacing Cys at positions 10 and 15 (numbering referring to the native H2R A peptide sequence throughout herein), Asp replacing Cys at positions 11 and 24. This compound also comprises H2R peptide B, the N-terminal 6 amino acids deleted, the 5 C-terminal amino acids deleted, the Cys at positions 11 and 23 (numbering referring to the native H2R B peptide sequence, throughout herein) are replaced with Dap, the 2-amino group and the carboxylic acid peptide (amide) bound within the peptide chain. The pendant 3-amino groups of the Dap in the H2R B peptide and the carboxyl groups of the Asp in the H2R A peptide are bonded via amide bonds. In another embodiment, the orientation of one or both of the bioisosteric bridges are reversed; i.e., the Dap may be in the H2R A peptide and the Asp in the H2R B peptide, for one or both bridges. In other embodiments, conservative or non conservative amino acid substitutions of one or more residues in either the H2R A peptide, the H2R B peptide, or both, are provided.

In one embodiment, the compound has the structure of Compound 3 below:

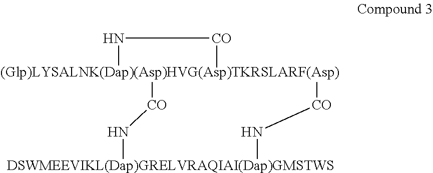

wherein Dap represents L-2,3-diaminopropionic acid. This compound comprises the H2R A peptide of H2R with an N-terminal Glp, Dap replacing Cys at position 10, Asp replacing Cys at positions 11, 15 and 24. This compound also comprises H2R peptide B, the Cys at positions 11 and 23 are replaced with Dap, the 2-amino group and the carboxylic acid peptide (amide) bound to the peptide chain. The pendant 3-amino group of the Dap at position 10 in the H2R A peptide and the carboxyl group of the Asp at position 15 of the H2R A peptide are intramolecularly bonded via amide bonds. The pendant 3-amino groups of the Dap at positions 11 and 23 of the H2R B peptide and the carboxyl groups of the Asp in the H2R A peptide at positions 11 and 24 are intermolecularly bonded via amide bonds. In another embodiment, the orientation of one or both of the bioisosteric intermolecular bridges are reversed; i.e., the Dap may be in the H2R A peptide and the Asp in the H2R B peptide, for one or both bridges. In other embodiments, conservative or non-conservative amino acid substitutions of one or more residues in either the H2R A peptide, the H2R B peptide, or both, are provided.

In one embodiment, the compound has the structure of Compound 4 below:

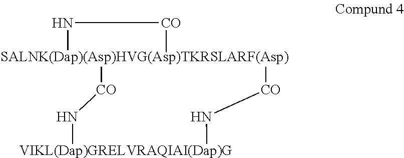

wherein Dap represents L-2,3-diaminopropionic acid. This compound comprises the H2R A peptide of H2R with a deletion of the first three amino acids, Dap replacing Cys at position 10, Asp replacing Cys at positions 11, 15 and 24. This compound also comprises H2R peptide B with a deletion of the first 6 amino acids and the last 5 amino acids, the Cys at positions 11 and 23 are replaced with Dap, the 2-amino group and the carboxylic acid peptide (amide) bound to the peptide chain. The pendant 3-amino group of the 2,3-diaminopropionic acid at position 10 in the H2R A peptide and the carboxyl group of the Asp at position 15 of the H2R A peptide are intramolecularly bonded via amide bonds. The pendant 3-amino groups of the Dap at positions 11 and 23 of the H2R B peptide and the carboxyl groups of the Asp in the H2R A peptide at positions 11 and 24 are bonded via amide bonds. In another embodiment, the orientation of one or both of the bioisosteric intermolecular bridges are reversed; i.e., the Dap may be in the H2R A peptide and the Asp in the H2R B peptide, for one or both bridges. In other embodiments, conservative or non-conservative amino acid substitutions of one or more residues in either the H2R A peptide, the H2R B peptide, or both, are provided.

In one embodiment, the compound has the structure of Compound 5 below:

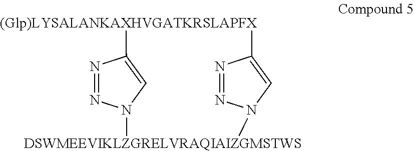

wherein the N-terminal amino acid of the H2R. A peptide is pyroglutamate, the Cys at positions 10 and 15 are replaced with Ala, and the Cys at positions 11 and 24 are replaced with propargylglycine (X). This compound also comprises H2R B peptide with the Cys at positions 11 and 23 replaced by 2-amino-4-azidobutyric acid (Z), with their pendant groups forming triazole rings, in accordance with Meldal et al., Angew. Chem. 2011; 123:5310-12. In other embodiments, one of both of the bioisosteric intermolecular bridges are reversed; i.e., the Cys at either position in the H2R A peptide may be replaced by 2-amino-4-azidobutyric acid (Z) and the Cys at positions 11 or 23 replaced with propargylglycine (X). In other embodiments, conservative or non-conservative amino acid substitutions of one or more residues in either the H2R A peptide, the H2R B peptide, or both, are provided.

In one embodiment, the compound has the structure of Compound 6 below:

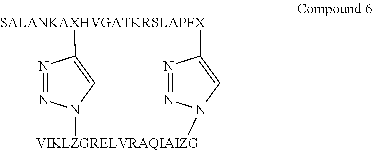

wherein the three N-terminal amino acids of the H2R A peptide are deleted, the Cys at positions 10 and 15 are replaced with Ala, and the Cys at positions 11 and 24 are replaced with propargylglycine (X). This compound also comprises H2R B peptide with the N-terminal 6 amino acids deleted, the C-terminal 5 amino acids deleted, the Cys at positions 11 and 23 replaced by 2-amino-4-azidobutyric acid (Z), and their pendant groups forming triazole rings, in accordance with Meldal et al., Angew. Chem. 2011; 123:5310-12. In other embodiments, one of both of the bioisosteric intermolecular bridges are reversed; i.e., the Cys at either position in the H2R A peptide may be replaced by 2-amino-4-azidobutyric acid (Z) and the Cys at positions 11 or 23 replaced with propargylglycine (X). In other embodiments, conservative or non-conservative amino acid substitutions of one or more residues in either the H2R A peptide, the H2R B peptide, or both, are provided.

In one embodiment, the compound has the structure of Compound 7 below:

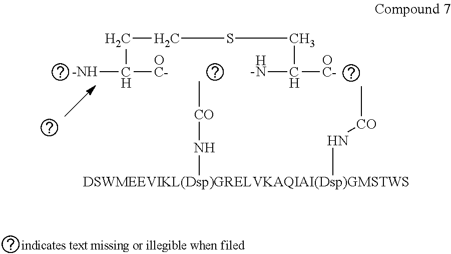

wherein Dap represents L-2,3-diaminopropionic acid and Z is Glu or Gln. This compound comprises the H2R A peptide of II2R with a cystathionine bridging positions 10 and 15, and an Asp replacing Cys at positions 11 and 24. This compound also comprises H2R B peptide, the Cys at positions 11 and 23 are replaced with Dap, the 2-amino group and the carboxylic acid peptide (amide) bound in the peptide chain. The pendant 3-amino groups of the 2,3-diaminopropionic acids at positions 11 and 23 of the H12R B peptide and the carboxyl groups of the Asp in the H2R A peptide at positions 11 and 24 are bonded via amide bonds. In another embodiment, the orientation of one or both of the bioisosteric intermolecular bridges are reversed; i.e., the Dap may be in the H2R A peptide and the Asp in the H2R B peptide, for one or both bridges. In other embodiments, conservative or non-conservative amino acid substitutions of one or more residues in either the H2R A peptide, the H2R B peptide, or both, are provided.

In one embodiment, the compound is the structure of Compound 8 below:

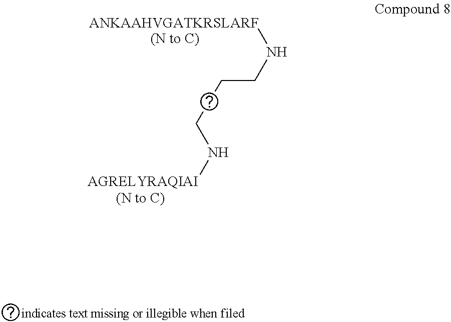

wherein the compound comprises the H2R A peptide with the N-terminal 6 amino acids deleted, the Ala replacing Cys at positions 10, 11 and 15, and the C-terminal Cys deleted. This compound also comprises H2R B peptide with the N-terminal 10 amino acids deleted, the Cys at position 11 replaced with Ala, and the C-terminal 7 amino acids deleted. The C-terminal carboxylic acids of the Phe of the H2R A peptide and the C-terminal Ile of the H2R B peptide are linked through amide bonds with 1,3-diaminopropane. In other embodiments, conservative or non-conservative amino acid substitutions of one or more residues in portions of either the H2R A peptide, the H2R B peptide, or both, present in Compound 8, are provided.

In one embodiment, the compound is the structure of Compound 9 below:

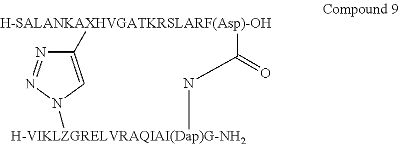

wherein the N-terminal 3 amino acids of the H2R A peptide are deleted, the Cys at positions 10 and 15 are replaced with Ala, and the Cys at position 11 is replaced with propargylglycine (X), and the Cys at position 24 is replaced with Asp. This compound also comprises H2R B peptide with 6 N-terminal amino acids deleted and the 5 C-terminal amino acids deleted, the Gly at position 24 having a C-terminal amide. The Cys at position 11 is replaced by 2-amino-4-azidobutyric acid (Z) and the Cys at position 23 replaced with Dap; the pendant groups of the propargylglycine (X) and 2-amino-4-azidobutyric acid (Z) forming a triazole ring, in accordance with Meldal et al., Angew. Chem. 2011; 123:5310 12, and the groups from the Asp and Dap forming an amide bond. In other embodiments, one of both of the bioisosteric intermolecular bridges are reversed; i.e., the Cys at position 11 in the H2R A peptide may be replaced by 2-amino-4-azidobutyric acid (Z) and the Cys at positions 11 replaced with propargylglycine (X); likewise the Asp and Dap replacing the other Cys may be reversed. In other embodiments, conservative or non-conservative amino acid substitutions of one or more residues in either the H2R A peptide, the H2R B peptide, or both, are provided.

In one embodiment, the compound is the structure of Compound 10 below:

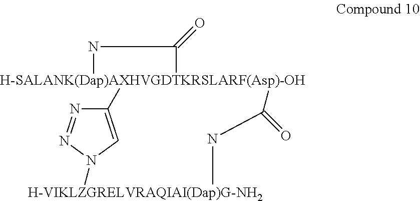

which incorporates similar features of Compound 9 but wherein a Dap is inserted before the Cys at position 10 of the A peptide, the Cys at position 10 is replaced with an Ala, the Cys at position 11 is replaced with a propargylglycine (X), and the Cys at position 15 is replaced with an Asp, and the Dap bonded intramolecularly to the Asp via an amide bond as shown in the structure above. Thus, the N-terminal 3 amino acids of the H2R A peptide are deleted, a Dap is inserted before the Cys at position 10, the Cys at position 10 is replaced with Ala, the Cys at position 11 replaced with a propargylglycine (X), and the Cys at position 24 is replaced with Asp. This compound also comprises H2R B peptide with 6 N-terminal amino acids deleted and the 5 C-terminal amino acids deleted, the Gly at position 24 having a C-terminal amide. The Cys at position 11 is replaced by 2-amino-4-azidobutyric acid (Z) and the Cys at position 23 replaced with Dap; the pendant groups of the propargylglycine (X) and 2-amino-4-azidobutyric acid (Z) forming a triazole ring, in accordance with Meldal et al., Angew. Chem. 2011; 123:5310-12, and the groups from the Asp and Dap forming an amide bond. In other embodiments, one of both of the bioisosteric intermolecular bridges are reversed; i.e., the propargylglycine (X) at position 11 in the H2R A peptide may be replaced by 2-amino-4-azidobutyric acid (Z), and the 2-amino-4-azidobutyric acid (Z) at position 11 in the H2R B peptide replaced with propargylglycine (X); likewise the Asp and Dap replacing the Cys at position 24 of the A peptide and Cys at position 23 of the B peptide, respectively, may be reversed. In other embodiments, conservative or non-conservative amino acid substitutions of one or more residues in either the II2R A peptide, the H2R B peptide, or both, are provided.

In other embodiments, the isolated peptide has less than 90%, less than 85%, less than 80%, less than 75%, less than 70%, less than 65%, less than 60%, less than 55%, less than 50%, less than 45%, less than 40%, less than 35%, less than 30%, or less than 20 percent sequence identity with any portion of the amino acid sequence of either the H2R A peptide or the H2R B peptide of human relaxin, or of both peptides.

In further embodiments, compositions including pharmaceutical compositions of the aforementioned compounds are embraced herein. In other embodiments, the invention also encompasses methods for treating, preventing or ameliorating a disease or disorder and or treating, restoring or ameliorating a tissue injury using relaxin-like peptides of the current invention. The invention also encompasses methods for treatment of heart failure and liver, lung and kidney fibrosis, among other injuries and diseases. In another embodiment, the compounds of the invention are useful for preventing, treating or reversing various fibrotic disorders, such as but not limited to fibrotic liver disease; hepatic ischemia-reperfusion injury; cerebral infarction: ischemic heart disease; renal disease; lung (pulmonary) fibrosis; liver fibrosis associated with hepatitis C, hepatitis B, delta hepatitis, chronic alcoholism, non-alcoholic steatohepatitis, stones in the bile duct, cholangiopathies selected from primary biliary cirrhosis and sclerosing cholangitis, autoimmune hepatitis, and inherited metabolic disorders selected from Wilson's disease, hemochromatosis, and alpha-lantitrypsin deficiency: damaged and/or ischemic organs, transplants or grafts; ischemia/reperfusion injury; stroke; cerebrovascular disease; myocardial ischemia; atherosclerosis; renal failure; renal fibrosis; idiopathic pulmonary fibrosis; wounds; ischemia/reperfusion injury in the brain, heart, liver and kidney; myocardial perfusion as a consequence of chronic cardiac ischemia or myocardial infarction; vascular occlusion; liver fibrosis or cirrhosis; radiocontrast nephropathy: fibrosis secondary to renal obstruction; renal trauma and transplantation; renal failure secondary to chronic diabetes and/or hypertension; and/or diabetes mellitus

### Synthesis of Compounds

Compounds of the current invention may be made using recombinant or synthetic techniques well known in the art. In particular, solid phase peptide/protein synthesis is well suited to the relatively short length of the H2R A peptides and H2R B peptides and may provide greater yields with more consistent results. The formation of the intramolecular and intermolecular bioisosteric bridges can be formed by methods known in the art and as described in the examples below. Additionally, the solid phase peptide/protein synthesis may provide additional flexibility regarding the manufacture of the individual peptides.

In solid-phase synthesis of H2R A and B peptides, an amino acid with both α-amino group and side chain protection is immobilized on a resin. See e.g. Nilsson, B., Soellner, M., and Raines, R. Chemical Synthesis of Proteins, Annu. Rev. Biomol. Struct. 2005. 34:91-118; Meldal M. 1997. Properties of solid supports. Methods Enzymol. 289:83-104 and Songster M F, Barany G. 1997. Handles for solid-phase peptide synthesis. Methods Enzymol. 289:126-74. Typically, two types of α-amino-protecting groups are used: an acid-sensitive tert-butoxycarbonyl (Boc) group or a base-sensitive 9-fluorenylmethyloxycarbonyl (Fmoc) group. Wellings D A, Atherton E. 1997. Standard Fmoc protocols. Methods Enzymol. 289:44-67. After the quick and complete removal of these α-amino-protecting groups another protected amino acid with an activated carboxyl group can then be coupled to the unprotected resin-bound amine. By using an excess of activated soluble amino acid, the coupling reactions are forced to completion. The cycle of deprotection and coupling is repeated to complete the sequence. With side chain deprotection and cleavage, the resin yields the desired peptide. Guy C A, Fields G B. 1997. Trifluoroacetic acid cleavage and deprotection of resin-bound peptides following synthesis by Fmoc chemistry. Methods Enzymol. 289:67-83, and Stewart J M. 1997. Cleavage methods following Boc-based solid-phase peptide synthesis. Methods Enzymol. 289:29-44. Additional methods for performing solid phase protein synthesis are disclosed in Bang, D. & Kent, S. 2004. A One-Pot Total Synthesis of Crambin. Angew. Chem. Int. Ed. 43:2534-2538; Bang, D., Chopra, N., & Kent, S. 2004. Total Chemical Synthesis of Crambin. J. Am. Chem. Soc. 126.1377-1383, Dawson, P. et al. 1994. Synthesis of Proteins by Native Chemical Ligation, Science. 266:776-779; Kochendoerfer et al. 2003. Design and Chemical Synthesis of a Homogenous Polymer-Modified Erythropoiesis Protein. Science. 299: 884-887. (Each reference recited in this paragraph is hereby incorporated by reference in its entirety.) The Dap, propargylglycine and 2-amino-4-azidobutyric acid, among other compounds may be incorporated into these peptides during synthesis as well.

If necessary, smaller peptides derived from solid phase peptide synthesis may be combined through peptide ligations such as native chemical ligation. In this process, the thiolate of an N-terminal cysteine residue of one peptide attacks the C-terminal thioester of a second peptide to affect transthioesterification. An amide linkage forms after rapid S→N acyl transfer. See Dawson, P. et al. 1994. Synthesis of Proteins by Native Chemical Ligation. Science. 266:776-779, which is hereby incorporated by reference in its entirety.

Further, one of ordinary skill in the art would recognize that the peptides of the compounds of the current invention may encompass peptidomimetics, peptides including both naturally occurring and non-naturally occurring amino acids, such as peptoids. Peptoids are oligomers of N-substituted glycines, glycoholic acid, thiopronine, sarcosine, and thiorphan. These structures tend to have a general structure of (—(C═O)—CH₂—NR—)ₙ with the R group acting as the side chain. Such peptoids can be synthesized using solid phase synthesis in accordance with the protocols of Simon et al., Peptoids: A molecular approach to drug discovery, Proc. Natl. Acad. Sci USA, 89:9367-9371 (1992) and Li et al., Photolithographic Synthesis of Peptoids, J. Am. Chem. Su. 2004, 126, 4088-4089, each of which is hereby incorporated by reference in its entirety. Additionally, the current invention contemplates the use of peptidomimetics or peptide mimetics, non-peptide drugs with properties analogous to those of the template peptide. (Fauchere, J. (1986) Adv. Drug Res. 15:29; Veber and Friedinger (1985) TINS p. 32; and Evans et al. (1987) J. Med. Chem 30:1229, which are incorporated by reference). Synthesis of various types of peptidomimetics has been reviewed for example in: Methods of Organic Chemistry (Houben-Weyl), Synthesis of Peptides and Peptidomimetics—Workbench Edition Volume E22c (Editor-in-Chief Goodman M.) 2004 (George Thieme Verlag Stuttgart, N.Y., hereby incorporated by reference in its entirety).

In one example, each peptide is assembled using an Fmoc/tBu strategy using 2-CTC resin. One resin aliquot was pre-loaded with 1,3-diaminopropane. The protected peptides were each released from the resin using 1% TFA in DCM and immediately neutralized. The two protected fragments were combined in stoichiometric amounts in DMF and amide formation was facilitated with DPPA to minimize racemization.

In addition to solid phase or liquid phase peptide synthesis, a variety of host-expression vector systems may be utilized to produce the peptides of the invention. Such host-expression systems represent vehicles by which the peptide of interest may be produced and subsequently purified, but also represent cells that may, when transformed or transfected with the appropriate nucleotide coding sequences, exhibit the modified erythropoietin gene product in situ. These include but are not limited to, bacteria, insect, plant, mammalian, including human host systems, such as, but not limited to, insect cell systems infected with recombinant virus expression vectors (e.g., baculovirus) containing the peptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing erythropoietin-related molecule coding sequences; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter.

In addition, a host cell strain may be chosen that modulates the expression of the inserted sequences, or modifies and processes the gene product in the specific fashion desired. Such modifications and processing of protein products may be important for the function of the protein. As known to those of ordinary skill in the art, different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and WI38.

For long-term, high-yield production of recombinant peptides, stable expression is preferred. For example, cell lines that stably express the recombinant gene product may be engineered. Rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the tissue-protective product. Such engineered cell lines may be particularly useful in screening and evaluation of compounds that affect the endogenous activity of the EPO-related molecule gene product.

Additional modifications can be made to the peptides. For example, the peptide may be synthesized with one or more (D)-amino acids. The choice of including an (L)- or (D)-amino acid into an H2R peptide of the present invention depends, in part, upon the desired characteristics of the peptide. For example, the incorporation of one or more (D)-amino acids can confer increasing stability on the peptide in vitro or in vivo. The incorporation of one or more (D)-amino acids can also increase or decrease the binding activity of the peptide as determined, for example, using the bioassays described herein, or other methods well known in the art.

Replacement of all or part of a sequence of (L)-amino acids by the respective sequence of entatiomeric (D)-amino acids renders an optically isomeric structure in the respective part of the polypeptide chain. Inversion of the sequence of all or part of a sequence of (L)-amino acids renders retro-analogues of the peptide. Combination of the enantiomeric (L to D, or D to L) replacement and inversion of the sequence renders retro-inverso-analogues of the peptide. It is known to those skilled in the art that enantiomeric peptides, their retro-analogues, and their retro-inverso-analogues maintain significant topological relationship to the parent peptide, and especially high degree of resemblance is often obtained for the parent and its retro-inverso-analogues. This relationship and resemblance can be reflected in biochemical properties of the peptides, especially high degrees of binding of the respective peptides and analogs to a receptor protein. The synthesis of the properties of retro-inverso analogues of peptides have been discussed for example in Methods of Organic Chemistry (Houben-Weyl), Synthesis of Peptides and Peptidomimetics—Workbench Edition Volume E22c (Editor-in-chief Goodman M.) 2004 (George Thieme Verlag Stuttgart, N.Y.), and in references cited therein, all of which are hereby incorporated by reference herein in their entireties.

Amino acid “modification” refers to the alteration of a naturally occurring amino acid to produce a non-naturally occurring amino acid. Derivatives of the peptides of the present invention with non-naturally occurring amino acids can be created by chemical synthesis or by site specific incorporation of unnatural amino acids into polypeptides during biosynthesis, as described in Christopher J. Noren, Spencer J. Anthony-Cahill, Michael C. Griffith, Peter G. Schultz, 1989 Science, 244:182-188, hereby incorporated by reference herein in its entirety.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂—NH—, —CH₂S—, —CH₂—CH₂—, —CH═CH-(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p 267 (1983); Spatola, A. F., Vega Data (March 1983), Vol. 1. Issue 3, “Peptide Backbone Modifications” (general review); Morely, J. S., Trends Pharma Sci (1980) pp. 463-468 (general review); Hudson, D. et al., (1979) Int J Pept Prot Re 14: 177-185 (—CH₂—NH—, —CH₂—CH₂—); Spatola, A. F. et al., (1986) Life Sci 38:1243-1249 (—CH₂—S); Hann, M. M., (1982) J Chem Soc Perkin Trans 1 307-314 (—CH═CH—, cis and trans); Almquist, R. G. et al., (1980) J Med Chem 23: 1392 (—COCH₂—); Jennings-White, C et al., (1982) Tetrahedron Lett 23:2533 (—COCH₂—); Szelke, M et al., European Appln. EP 45665 (1982) CA: 97: 39405 (1982) (—CH(OH)CH₂—); Holladay, M. W. et al., (1983) Tetrahedron Lett 24:4401-4404 (—C(OH)CH₂—); and Hruby, V. J., (1982) Life Sci 31:189-199 (—CH₂—S—); each of which is incorporated herein by reference.

In another embodiment, a particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

A variety of designs for peptide mimetics are possible. For example, cyclic peptides, in which the necessary conformation is stabilized by non-peptides, are specifically contemplated, U.S. Pat. No. 5,192,746 to Lobl, et al., U.S. Pat. No. 5,576,423 to Aversa, et al., U.S. Pat. No. 5,051,448 to Shashoua, and U.S. Pat. No. 5,559,103 to Gaeta, et al., all hereby incorporated by reference, describe multiple methods for creating such compounds. Synthesis of nonpeptide compounds that mimic peptide sequences is also known in the art. Eldred et al., J. Med. Chem. 37:3882 (1994), hereby incorporated by reference herein in its entirety) describe non-peptide antagonists that mimic the peptide sequence. Likewise, Ku et al., J. Med. Chem 38:9 (1995) (hereby incorporated by reference herein in its entirety) further elucidates the synthesis of a series of such compounds.

Either conservative or non-conservative amino acid substitutions can be made at one or more amino acid residues. Both conservative and non-conservative substitutions can be made at different positions. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar (hydrophobic)-cysteine, alanine, valine, leucine, isoleucine, proline, phenylalanine, methionine, tryptophan, glycine, tyrosine; and (4) uncharged polar=asparagine, glutamine, serine, threonine. Non-polar may be subdivided into: strongly hydrophobic=alanine, valine, leucine, isoleucine, methionine, phenylalanine and moderately hydrophobic=glycine, proline, cysteine, tyrosine, tryptophan. In alternative fashion, the amino acid repertoire can be grouped as (1) acidic=aspartate, glutamate; (2) basic=lysine, arginine, histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, W H Freeman and Co., 1995, which is incorporated by reference herein in its entirety). Non-conservative substitutions embody changing an amino acid to another that is not in the same family as described above; the biological activity of a relaxin mimetic of the invention incorporating one or more non-conservative substitutions can be readily assessed using the assays described herein.

Alternatively, mutations can be introduced randomly along all or part of the coding sequence of H2R A or B peptide, such as by saturation mutagenesis, and the resultant mutants can be screened for biological activity to identify mutants that retain activity. Following mutagenesis, the encoded peptide can be expressed recombinantly and the activity of the recombinant peptide can be determined.

In another embodiment, the peptide may be further modified through the additions of polymers (such as polyethylene glycol), sugars, or additional proteins (such as a fusion construct) in an effort to extend the half-life of the peptide or enhance the peptide's activities.

### Biological Screens or Assays

Relaxin-like peptides in accordance with the present invention may be tested for biological activity by any from among several in vitro and in vivo assays, as described below. These are merely exemplary and non-limiting, and the literature described herein and others well known to the skilled artisan can be followed to assess biological activity.

Receptor Binding & Functional Assay: Compounds can be assayed for their ability to bind the RXFP1 receptor (competition assay using labeled H2R) and generate cAMP in HEK293 cells transfected with the H2R receptor.

Antifibrotic Activity: To rapidly identify those compounds with antifibrotic activity, an assay comprising human hepatic stellate cells (HSCs) that express RXFP1 can be used. Cells are plated in 6-well tissue culture plates and after 3 days switched to starvation medium. Three days later, cells are challenged with TGFβ1 (10 ng/ml) in the presence of vehicle or peptides (100 ng/ml, n=3). Soluble collagen in the supernatant is quantitated after 72 hr using the commercially available Sircol kit. Compounds of the invention typically markedly reduced TGFβ1-driven collagen production.

In Vivo Activity. Antifibrotic effects of selected H2R-like peptides can be evaluated in the murine ureteral obstruction (UUO) model—an accelerated, highly aggressive and reproducible model of primary tubulointerstitial fibrosis that occurs independently of species and strain, without the confounding variable of hypertension and demonstrates changes that mimic the pathology of human progressive renal disease. Adult male C57BL/6 mice are subjected to left UUO and randomized 4 hr later to vehicle or peptides (n>7; 500 ug/kg/day s.c.; Alzet 1007D, 0.5 ml/hr to achieve circulating peptide concentrations of ˜40 ng/ml). H2R (500 ug/kg/day s.c.; n=3) is used as positive control. Mice are sacrificed 4 days following UUO and kidneys analyzed for renal hydroxyproline content, a marker of renal collagen accumulation.

Animal model systems can be used to demonstrate the relaxin-like activity of a compound or to demonstrate the safety and efficacy of the compounds identified by the screening methods of the invention described above. The compounds identified in the assays can then be tested for biological activity using animal models for a type of tissue damage, disease, condition, or syndrome of interest.

### Therapeutic Uses

One of ordinary skill in the art would readily recognize that the compounds of the current invention are useful as therapeutics for treatment or prevention of various diseases, disorders, and conditions. Both in vitro and in vivo techniques that can be used for assessing the therapeutic indications of, for example, the compounds identified by the inventive assays disclosed above.

In one embodiment, such a pharmaceutical composition comprising a compound can be administered systemically to protect or enhance the target cells, tissue or organ. Such administration may be parenterally, via inhalation, or transmucosally, e.g., orally, nasally, rectally, intravaginally, sublingually, ocularly, submucosally or transdermally. Preferably, administration is parenteral, e.g., via intravenous or intraperitoneal injection, and also including, but is not limited to, intra-arterial, intramuscular, intradermal and subcutaneous administration.

For other routes of administration, such as by use of a perfusate, injection into an organ, or other local administration, a pharmaceutical composition will be provided which results in similar levels of a compound as described above. A level of about 15 pM −30 nM is preferred.

The pharmaceutical compositions of the invention may comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin, hereby incorporated by reference herein in its entirety. Such compositions will contain a therapeutically effective amount of the compound, preferably in purified form, together with a suitable amount of carrier so as to provide the form for proper administration to the patient. The formulation should suit the mode of administration.

Formulations for increasing transmucosal adsorption of peptides such as long acting peptides are also contemplated by the current invention. Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation of compounds directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention ale administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-scaled container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A perfusate composition may be provided for use in transplanted organ baths, for in situ perfusion, or for administration to the vasculature of an organ donor prior to organ harvesting. Such pharmaceutical compositions may comprise levels of peptides, or a form of peptides not suitable for acute or chronic, local or systemic administration to an individual, but will serve the functions intended herein in a cadaver, organ bath, organ perfusate, or in situ perfusate prior to removing or reducing the levels of the peptide contained therein before exposing or returning the treated organ or tissue to regular circulation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, for example, H2R-like peptide can be delivered in a controlled-release system. For example, the peptide may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574, each of which is incorporated by reference herein in its entirety). In another embodiment, the compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); WO 91/04014; U.S. Pat. No. 4,704,355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard et al., 1989, J. Neurosurg. 71:105, (each of which is incorporated by reference herein in its entirety).

In yet another embodiment, a controlled release system can be placed in proximity of the therapeutic target, i.e., the target cells, tissue or organ, thus requiring only a fraction of the systemic dose (see, e.g., Goodson, pp. 115-138 in Medical Applications of Controlled Release, vol. 2, supra, 1984, which is incorporated by reference herein in its entirety). Other controlled release systems are discussed in the review by Langer (1990, Science 249:1527-1533, which is incorporated by reference herein in its entirety).

In another embodiment, peptide, as properly formulated, can be administered by nasal, oral, rectal, vaginal, ocular, transdermal, parenteral or sublingual administration.

In a specific embodiment, it may be desirable to administer H2R-like peptide of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. A non-limiting example of such an embodiment would be a coronary stent coated with H2R-like peptide of the present invention.

Selection of the preferred effective dose will be readily determinable by a skilled artisan based upon considering several factors, which will be known to one of ordinary skill in the art. Such factors include the particular form of peptide, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

In another aspect of the invention, a perfusate or perfusion solution is provided for perfusion and storage of organs for transplant, the perfusion solution includes an amount of H2R-like peptide effective to protect responsive cells and associated cells, tissues or organs. Transplant includes but is not limited to allotransplantation, where an organ (including cells, tissue or other bodily part) is harvested from one donor and transplanted into a different recipient, both being of the same species; autotransplantation, where the organ is taken from one part of a body and replaced at another, including bench surgical procedures, in which an organ may be removed, and while ex vivo, resected, repaired, or otherwise manipulated, such as for tumor removal, and then returned to the original location or xenotransplantation, where tissues or organs or transplanted between species. In one embodiment, the perfusion solution is the University of Wisconsin (UW) solution (U.S. Pat. No. 4,798,824, hereby incorporated by reference herein in its entirety) which contains from about 1 to about 25 U/ml (10 ng=1 U) of compound, 5% hydroxyethyl starch (having a molecular weight of from about 200,000 to about 300,000 and substantially free of ethylene glycol, ethylene chlorohydrin, sodium chloride and acetone); 25 mM KH₂PO₄; 3 mM glutathione; 5 mM adenosine; 10 mM glucose; 10 mM HEPES buffer; 5 mM magnesium gluconate; 1.5 mM CaCl₂; 105 mM sodium gluconate; 200,000 units penicillin; 40 units insulin; 16 mg dexamethasone; 12 mg Phenol Red; and has a pH of 7.4-7.5 and an osmolality of about 320 mOsm/l. The solution is used to maintain cadaveric kidneys and pancreases prior to transplant. Using the solution, preservation can be extended beyond the 30-hour limit recommended for cadaveric kidney preservation. This particular perfusate is merely illustrative of a number of such solutions that can be adapted for the present use by inclusion of an effective amount of H2R-like peptide. In a further embodiment, the perfusate solution contains from about 1 to about 500 ng/ml of H2R-like peptide, or from about 40 to about 320 ng/ml peptide. As mentioned above, any form of peptide can be used in this aspect of the invention.

While the preferred recipient of H2R-like peptide for the purposes herein throughout is a human, the methods herein apply equally to other mammals, particularly domesticated animals, livestock, companion, and zoo animals. However, the invention is not so limiting and the benefits can be applied to any mammal.

In further aspects of the ex-vivo invention, any peptide such as but not limited to the ones described above may be employed.

H2R-like peptide of the invention may be administered systemically at a dosage between about 1 ng and about 100 μg/kg body weight, preferably about 5-50 μg/kg-body weight, most preferably about 10-30 μg/kg-body weight, per administration. This effective dose should be sufficient to achieve serum levels of peptides greater than about 80, 120, or 160 ng/ml of serum after administration. Such serum levels may be achieved at about 1, 2, 3, 4, 5, 6, 7, 8, 9, or 10 hours post-administration. Such dosages may be repeated as necessary. For example, administration may be repeated daily, as long as clinically necessary, or after an appropriate interval, e.g., every 1 to 12 weeks, preferably, every 1 to 3 weeks. In one embodiment, the effective amount of peptide and a pharmaceutically acceptable carrier may be packaged in a single dose vial or other container.

### EXAMPLES

### Example 1

### Method of Peptide Synthesis

Each peptide was assembled using an Fmoc/tBu strategy using 2-CTC resin. One resin aliquot was pre-loaded with 1,3-diaminopropane. The protected peptides were each released from the resin using 1% TFA in DCM and immediately neutralized. The two protected fragments were combined in stoichiometric amounts in DMF and amide formation was facilitated with DPPA to minimize racemization. The coupling goes very slowly due to the dilution required to get these sparingly soluble fragments in solution. A small micro sample was removed and cleaved with Reagent K in order to access the extent of coupling.

### Example 2

### Compounds of the Invention Stimulate Camp Via RXFP1

A cell line (HEK-293T cells) stably expressing RXFP1 (HEK-RXFP1 cells) was used. Parallel experiments were performed with the parental cells (HEK-293T cells), which do not express RXFP1 and do not display increased cAMP in response to relaxin. Cells were sccdcd at 20,000 cells per well in PBS buffer and treated with relaxin (10 ng/mL) or compound of the invention (10 g/mL) for 30 or 60 min. The production of cAMP was determined using the cAMP-Glo assay (Promega, Madison Wis.), a luciferase-based assay. The level of cAMP in the cells was determined by comparison to a cAMP standard curve. Due The data are presented as absolute cAMP levels in the wells after subtraction of vehicle effects. There was no cAMP response to either relaxin or inventive compound in parental HEK-293T cells (which lack RXFP1).

Activation of cAMP in THP-1 cells. THP-1 cells were centrifuged to pellet, resuspended in PBS containing 0.5 mM isobutylmethylxanthine (IBMX) and plated into 96 well plates at 200,000 cells/rxn in 90 ul. The test compound was added to cells at a final concentration of 10000, 1000 or 100 ng/ml and cells incubated for 2 hours at 37 deg C. As controls, cells were incubated with human relaxin (100 ng/ml) or Forskolin (10 uM) as positive controls and cells alone as a negative/baseline control.

Following incubation, cAMP was measured using the commercially available cAMP-Go™ Assay (Promega) following the manufacturer's instructions. The data was plotted as ΔRLU's arrived at by subtracting test samples from the cells alone control (FIG. 2).

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A compound selected from among: 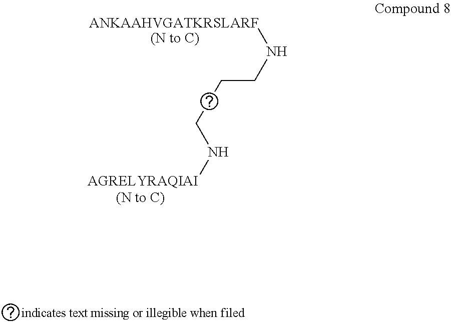
wherein the C-terminal carboxylic acids of the Phe of the H2R A peptide and the C-terminal Ile of the H2R B peptide are linked through amide bonds with 1,3-diaminopropane; 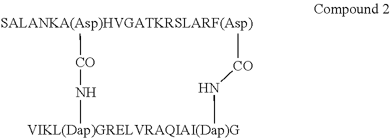
wherein Dap represents L-2,3-diaminopropionic acid; 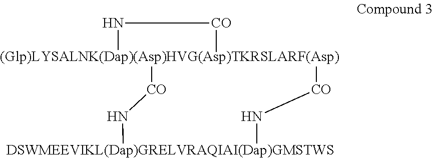
wherein Dap represents L-2,3-diaminopropionic acid and Glp represents pyroglutamate; 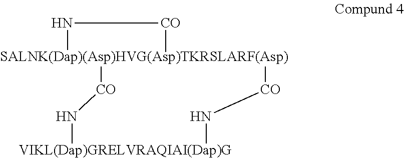
wherein Dap represents L-2,3-diaminopropionic acid; 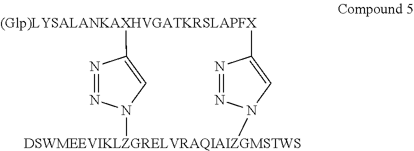
wherein Glp is pyroglutamate, X is propargylglycine and Z is 2-amino-4-azidobutyric acid, or Z is propargylglycine and X is 2-amino-4-azidobutyric acid; 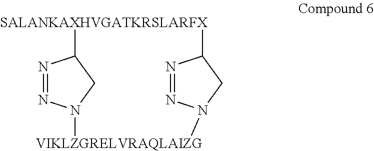
wherein X is propargylglycine and Z is 2-amino-4-azidobutyric acid, or Z is propargylglycine and X is 2-amino-4-azidobutyric acid; 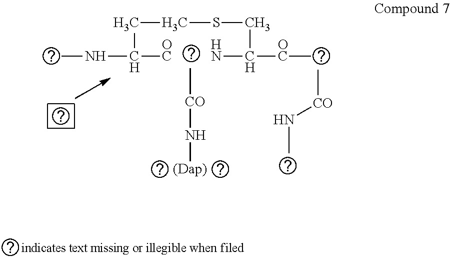
wherein Z is Glu or Gln; 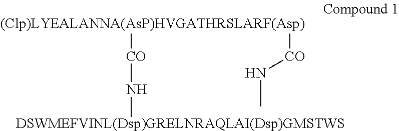
wherein Dap represents L-2,3-diaminopropionic acid; 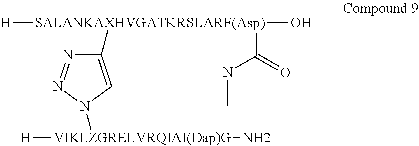
wherein Dap represents L-2,3-diaminopropionic acid, X is propargylglycine and Z is 2-amino-4-azidobutyric acid; and 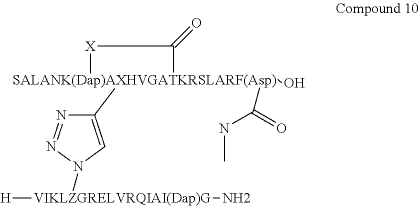
wherein Dap represents L-2,3-diaminopropionic acid, X is propargylglycine and Z is 2-amino-4-azidobutyric acid.

2. The compound of claim 1 wherein one or more amino acid is replaced with a conservative or a non-conservative substitution.

3. An acyclic or cyclic compound that comprises a modified H2R A peptide linked to a modified H2R B peptide, wherein one or more intramolecular or intramolecular cystines is replaced with a bioisosteric substitution.

4. The compound of claim 3 wherein the H2R A peptide, the H2R B peptide, or the combination thereof is truncated.

5. The compound of claim 3 wherein one or more cysteine residues in the peptide that are not replaced with a bioisostric substitution are replaced with another amino acid.

6. The compound of claim 5 wherein one or more cysteine residues in the peptide that is not replaced with a bioisosteric substitution is replaced with alanine.

7. The compound of claim 3 wherein the bioisosteric substitution is selection from among 1) a diaminopropane molecule bound via amide bonds to the C-terminal carboxylic acids of the A and H2R B peptides; 2) an aspartic acid or glutamic acid replacing one cysteine residue of a cystine bridge, and a L-2,3-diaminopropionic acid replacing the other cysteine residue of the cysteine bridge, the pendant amino group of the L-2,3-diaminopropionic acid and the carboxylic acid of the aspartic acid linked by an amide bond; 3) a propargylglycine replacing one cysteine residue of a cysteine bridge and a 2-amino-4-azidobutyric acid replacing the other cysteine residue of the cysteine bridge, the pendant groups of the two molecules forming a triazine ring; 4) a cystine bridge replaced by cystathionine, or any combination of any of the foregoing.

8. The compound of claim 3 wherein an N-terminal glutamine or glutamic acid is a pyroglutamate residue.

9. A compound of claim 3 selected from among 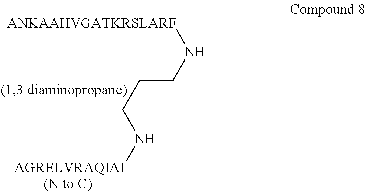
wherein the C-terminal carboxylic acids of the Phe of the H2R A peptide and the C-terminal Ile of the H2R B peptide are linked through amide bonds with 1,3-diaminopropane; 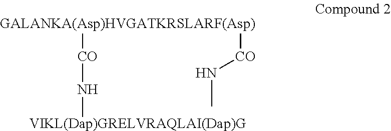
wherein Dap represents L-2,3-diaminopropionic acid; 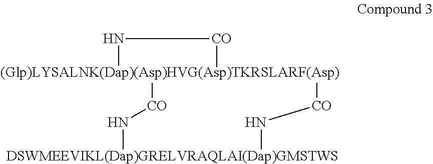
wherein Dap represents L-2,3-diaminopropionic acid and Glp represents pyroglutamate; 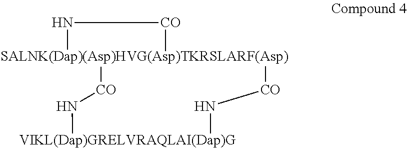
wherein Dap represents L-2,3-diaminopropionic acid; 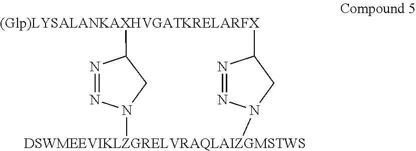
wherein Glp is pyroglutamate, X is propargylglycine and Z is 2-amino-4-azidobutyric acid, or Z is propargylglycine and X is 2-amino-4-azidobutyric acid; 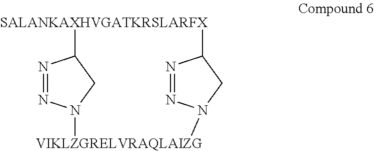
wherein X is propargylglycine and Z is 2-amino-4-azidobutyric acid, or Z is propargylglycine and X is 2-amino-4-azidobutyric acid; 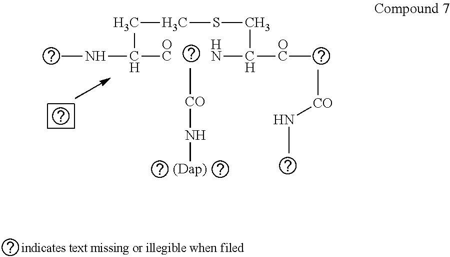
wherein Z is Glu or Gln; 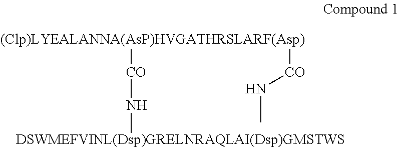
wherein Dap represents L-2,3-diaminopropionic acid; 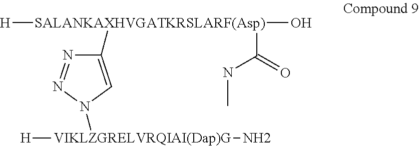
wherein Dap represents L-2,3-diaminopropionic acid, X is propargylglycine and Z is 2-amino-4-azidobutyric acid; and 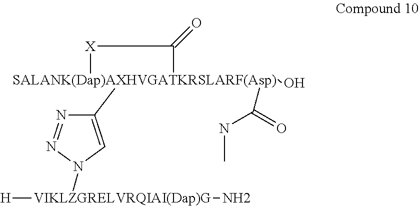
wherein Dap represents L-2,3-diaminopropionic acid, X is propargylglycine and Z is 2-amino-4-azidobutyric acid.

10. A compound of any one of claims 3-9 wherein one or more amino acid is replaced with a conservative or a non-conservative substitution.

11. A pharmaceutical composition comprising a compound of any one of claims 1-10 and a pharmaceutically acceptable carrier, excipient or diluent.

12. A method for preventing or treating an injury or disease comprising administering to a subject in need thereof an effective amount of a compound of any one of claims 1-10 or a pharmaceutical composition of claim 11, wherein the injury or disease is fibrotic liver disease; hepatic ischemia-reperfusion injury; cerebral infarction: ischemic heart disease; renal disease; lung (pulmonary) fibrosis; liver fibrosis associated with hepatitis C, hepatitis B, delta hepatitis, chronic alcoholism, non-alcoholic steatohepatitis, stones in the bile duct, cholangiopathies selected from primary biliary cirrhosis and sclerosing cholangitis, autoimmune hepatitis, and inherited metabolic disorders selected from Wilson's disease, hemochromatosis, and alpha-lantitrypsin deficiency, damaged and/or ischemic organs, transplants or grafts; ischemia/reperfusion injury; stroke; cerebrovascular disease; myocardial ischemia; atherosclerosis; renal failure; renal fibrosis; idiopathic pulmonary fibrosis; wounds; ischemia/reperfusion injury in the brain, heart, liver and kidney; myocardial perfusion as a consequence of chronic cardiac ischemia or myocardial infarction; vascular occlusion; liver fibrosis or cirrhosis; radiocontrast nephropathy: fibrosis secondary to renal obstruction; renal trauma and transplantation; renal failure secondary to chronic diabetes and/or hypertension; and/or diabetes mellitus.

13. The method of claim 12 wherein the compound or pharmaceutical composition thereof is administered parenterally, via inhalation, intranasally or orally.

14. The method of claim 13 wherein parenterally is by intravenous, intraperitoneal, intra-arterial, intramuscular, intradermal or subcutaneous administration.

15. The compound of any one of claims 1-10 that activates the RXFP1 receptor.

16. The compound of any one of claims 1-10 wherein the compound produces cAMP or NO.
